(19) United States
(12) Patent Application Publication
(10) Pub. No.: US 2026/0115445 A1
(43) Pub. Date: Apr. 30, 2026

(54) ASEPTIC CONNECTOR

(71) Applicant: WATSON-MARLOW LIMITED, Falmouth Cornwall (GB)

(72) Inventors: Izzy BILLAU, Falmouth (GB); Simon WOODS, Falmouth (GB); Nick WHITE, Falmouth (GB)

(73) Assignee: WATSON-MARLOW LIMITED, Falmouth Cornwall (GB)

(21) Appl. No.: 18/992,159

(22) PCT Filed: Jul. 6, 2023
(86) PCT No.: PCT/EP2023/068798

§ 371 Date: Jan. 7, 2025

(30) Foreign Application Priority Data
Jul. 8, 2022 (GB) 2210049.9

(51) Int. Cl. A61M 39/22; A61M 39/10; F16L 37/373

(52) U.S. Cl. CPC A61M 39/22; A61M 39/10; F16L 37/373; A61M 2039/226; A61M 2205/583

(57) ABSTRACT

There is described an aseptic connector for connection to an identical opposing aseptic connector, the aseptic connector comprising: a housing having an inner surface which defines a cavity, the housing having a fluid passageway which passes through the inner surface and forms an opening into the cavity; a rotatable member rotatably mounted within the cavity of the housing and having a fluid passageway extending therethrough; wherein the rotatable member has a first, closed configuration in which the fluid passageway of the rotatable member is sealed by the inner surface of the housing and a second, open configuration in which the fluid passageway of the rotatable member is aligned with the fluid passageway of the housing and extends to the fluid passageway of the housing of the opposing aseptic connector so as to form a continuous fluid pathway across the aseptic connectors.

### CROSS REFERENCE TO RELATED APPLICATIONS

This application is a U.S. national stage of PCT/EP2023/068798 filed Jul. 6, 2023 which claims priority to United Kingdom Patent Application 2210049.9 filed Jul. 8, 2022, the contents of which are incorporated herein.

### TECHNICAL FIELD

The disclosure relates to an aseptic connector.

### BACKGROUND

Sterile connectors for aseptic processing (commonly referred to as “aseptic connectors”) enable two lines of tubing to be joined while maintaining a sterile fluid pathway. This may be particularly important in medical, pharmaceutical and bioprocessing applications.

An end of each line of tubing may be provided with an aseptic connector which is configured to engage and mechanically connect with the opposing connector. Each aseptic connector is provided with a sealing arrangement which retains the sterility of the tubing (as well as any portions of the connector which form part of the fluid pathway) before, during and after their interconnection. For example, the end of each connector may be provided with a removable membrane which can be removed once the two connectors are interconnected, and the fluid pathway is sealed.

It is desirable to provide aseptic connectors which are simple and easy to operate in order to avoid user errors which could inadvertently compromise the sterility of the fluid pathway.

In accordance with an aspect of the disclosure, there is provided an aseptic connector for connection to an identical opposing aseptic connector, the aseptic connector comprising: a housing having an inner surface which defines a cavity, the housing having a fluid passageway which passes through the inner surface and forms an opening into the cavity; a rotatable member rotatably mounted within the cavity of the housing and having a fluid passageway extending therethrough; wherein the rotatable member has a first, closed configuration in which the fluid passageway of the rotatable member is sealed by the inner surface of the housing and a second, open configuration in which the fluid passageway of the rotatable member is aligned with the fluid passageway of the housing and extends to the fluid passageway of the housing of the opposing aseptic connector so as to form a continuous fluid pathway across the aseptic connectors.

One of the rotatable member and the housing may comprise a groove and the other of the rotatable member and the housing may comprise a rib which engages with the groove to rotatably mount the rotatable member within the housing.

The groove and/or rib may be semi-circular.

The rotatable member may comprise an actuator portion which extends externally to the housing so as to allow the rotatable member to be rotated between the first and second configurations.

The actuator portion may comprise a male element and a female element which respectively engage with the female element and the male element of the actuator portion of the opposing aseptic connector.

The housing of the aseptic connector may comprise a male element and a female element which respectively engage with the female element and the male element of the housing of the opposing aseptic connector.

The male and female elements of the housing may form snap fit connections with the female and male elements of the housing of the opposing aseptic connector.

The aseptic connector may further comprise a visual indicator which is visible only when the rotatable member is in the first configuration and/or a visual indicator which is visible only when the rotatable member is in the second configuration.

The or each visual indicator may be provided on the rotatable member and the housing may comprise a window through which the or each visual indicator can be seen.

A dividing bar may be provided across the opening of the fluid passageway; wherein the dividing bar seals against adjacent portions of the rotatable members of the aseptic connector and the opposing aseptic connector.

The dividing bar may comprise a leading edge which is configured to divide an incoming fluid flow between the fluid passageways of the aseptic connector and the opposing aseptic connector.

The rotatable member may comprise an external surface which faces outwardly when in the first configuration; wherein the external surface abuts and seals against the external surface of the rotatable member of the opposing aseptic connector such that the external surfaces do not form part of the fluid pathway.

The inner surface of the housing and the rotatable member of the aseptic connector may respectively combine with the inner surface of the housing and the rotatable member of the opposing aseptic connector to form a surface of revolution having circular symmetry.

The aseptic connector may further comprise a seal member disposed between the housing and the rotatable member; wherein the seal member has an opening which connects the fluid passageway of the housing with the fluid passageway of the rotatable member.

The seal member may be slidable between a first position in which the opening is offset from the fluid passageway of the housing and a second position in which the opening is aligned with the fluid passageway of the housing; wherein the seal member is slidable between the first and second positions when the rotatable member transitions between the first, closed configuration and the second, open configuration.

In the first, closed configuration the fluid passageway of the rotatable member may be sealed by the slidable seal member and the rotatable member may rotate relative to the slidable seal member during an initial rotation towards the second, open configuration in order to bring the fluid passageway of the rotatable member into alignment with the opening of the seal member.

The aseptic connector may further comprise a liner seal which is disposed between the housing and the rotatable member.

The liner seal may extend around a perimeter of the inner surface of the housing.

The aseptic connector may further comprise a seal which is located in a bore formed in the housing and which has a throughbore that forms part of the fluid passageway.

The seal may comprise first and second portions which each have a throughbore for connecting to the fluid passageway of the rotatable member of the aseptic connector and the rotatable member of the opposing aseptic connector.

The first and second portions may be integrally formed.

The first and second portions may be joined by a web.

The first and second portions may have hourglass-like profiles.

In accordance with another aspect of the disclosure, there is provided an aseptic connector assembly comprising a pair of aseptic connectors as described above.

### BRIEF DESCRIPTION OF DRAWINGS

For a better understanding of the disclosure, and to show more clearly how it may be carried into effect, reference will now be made, by way of example, to the accompanying drawings, in which:

FIG. 1 is a perspective view of an aseptic connector according to an embodiment of the disclosure;

FIGS. 2 and 3 are exploded views of the aseptic connector;

FIG. 4 is a rear perspective view of the aseptic connector in a first, closed configuration;

FIGS. 5 and 6 are front and rear perspective views of the aseptic connector in a second, open configuration;

FIGS. 7 and 8 show a pair of aseptic connectors in the first, closed configuration being brought into mechanical engagement;

FIG. 9 is cross-sectional view of the aseptic connectors in the position shown in FIG. 8;

FIG. 10 shows the aseptic connectors during a transition between the first, closed configuration and the second, open configuration;

FIG. 11 is a cross-sectional view of the aseptic connectors in the position shown in FIG. 10;

FIG. 12 shows the aseptic connectors in the second, open configuration;

FIG. 13 is a cross-sectional view of the aseptic connectors in the position shown in FIG. 12;

FIG. 14 is a rear perspective view of an aseptic connector according to another embodiment in a first, closed configuration;

FIG. 15 is cross-sectional view of the aseptic connector in the first, closed configuration;

FIG. 16 is a rear perspective view of a housing of the aseptic connector;

FIG. 17 is a rear perspective view of the aseptic connector in a second, open configuration;

FIG. 18 is an exploded view of an aseptic connector according to another embodiment;

FIG. 19 is a perspective cutaway view of a pair of aseptic connectors in a first, closed configuration;

FIG. 20 shows the pair of aseptic connectors during a transition between the first configuration and a second, open configuration;

FIG. 21 shows the pair of aseptic connectors in the second, open configuration;

FIG. 22 is a perspective view showing a housing of an aseptic connector according to another embodiment; and

FIG. 23 is a cross-sectional view of a pair of aseptic connectors in a second, open configuration.

### DETAILED DESCRIPTION

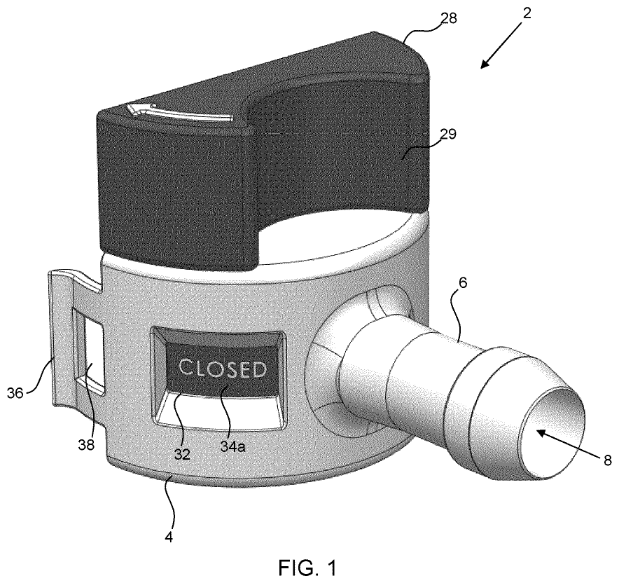

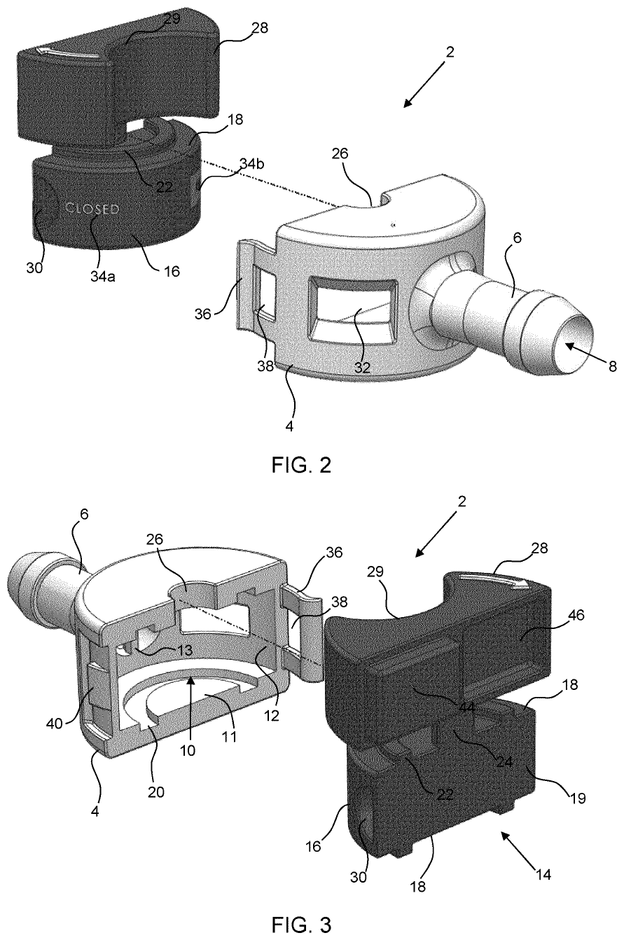

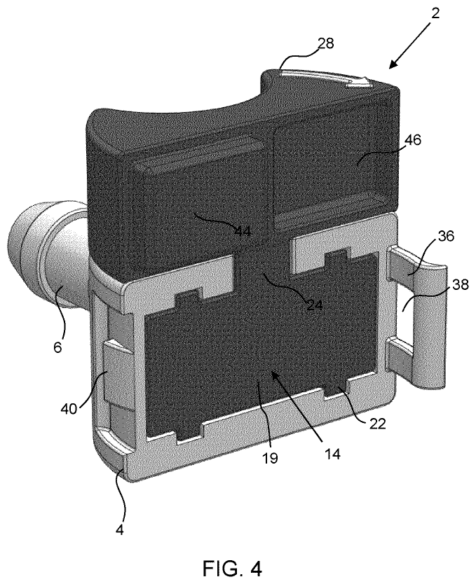

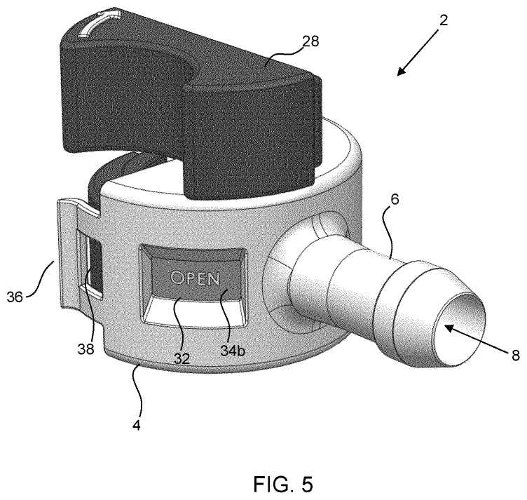

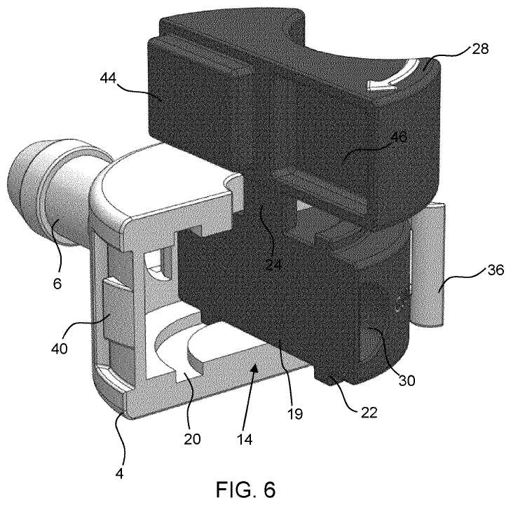

FIGS. 1 to 3 show an aseptic connector 2 according to an embodiment of the disclosure.

The aseptic connector 2 generally comprises a housing 4. The housing 4 comprises a barb fitting 6 for insertion into a tube. A fluid passageway 8 extends through the barb fitting 6. The housing 4 defines a semi-cylindrical inner cavity 10 which is bounded by a circumferential surface 12 and a pair of radial end surfaces 11. The fluid passageway 8 opens into the circumferential surface 12. The opening in the circumferential surface 12 is provided with a dividing bar 13 which extends across the diameter of the opening.

The inner cavity 10 receives a semi-cylindrical rotatable member 14 which comprises a circumferential surface 16, a pair of radial end surfaces 18 and an axial end surface 19. The semi-cylindrical rotatable member 14 is complementary to the semi-cylindrical inner cavity 10 such that it can be received within the inner cavity 10 with the circumferential surface 16 of the rotatable member 14 running parallel with (but slightly spaced from) the circumferential surface 12 of the inner cavity 10.

The radial end surfaces 11 of the housing 4 each comprise a semi-circular groove 20 and the radial end surfaces 18 of the rotatable member 14 each comprise a complementary semi-circular rib 22. The grooves 20 are open at either end and the ribs 22 of the rotatable member 14 can be rotated into engagement with the grooves 20 in order to engage the rotatable member 14 with the housing 4 but to allow rotation of the rotatable member 14 relative to the housing 4. The rotating engagement is therefore located away (i.e., radially outward) from the axis of rotation of the rotatable member 14.

The rotatable member 14 further comprises a central shaft 24 which extends from the centre of one of the radial end surfaces 18. The central shaft 24 is semi-cylindrical and its axial end surface forms an extension of the axial end surface 19. The housing 4 comprises a complementary semi-circular recess 26 which receives the central shaft 24 when the ribs 22 are engaged in the grooves 20.

The rotatable member 14 further comprises a passageway 30 which has an opening at either end at diametrically opposed positions in the circumferential surface 16 and extends through the rotatable member 14 in a direction which is parallel to the axial end surface 19.

An actuation knob 28 (or any other actuation portion) is connected to the central shaft 24 and is disposed externally to the housing 4. The actuation knob 28 can be grasped and rotated in order to rotate the rotatable member 14 within the inner cavity 10 between a first, closed position and a second, open position, as will be described in further detail below.

The circumferential surface 12 of the housing 4 comprises a window 32 through which the circumferential surface 16 of the rotatable member 14 can be seen. The circumferential surface 16 of the rotatable member 14 is provided with a first visual indicator 34a and a second visual indicator 34b. The first visual indicator 34a is positioned such that it is visible through the window 32 when the rotatable member is in the first, closed position and the second visual indicator 34b is positioned such that it is visible through the window 32 when the rotatable member is in the second, open position. As shown, in this example, the first visual indicator 34a comprises the word “CLOSED” on a red background, whereas the second visual indicator 34b comprises the word “OPEN” on a green background; however, it will be appreciated that in other examples alternative forms of visual indicator may be used. Accordingly, the window 32 and visual indicators 34a, 34b provide feedback to a user regarding the current state of the connector 2. In other examples, only a single visual indicator may be provided which confirms that the connector 2 is in one of the open and closed configurations and the absence of their visual indicator is used to demonstrate that the connector 2 is in the other of the open and closed configurations. Further, the or each visual indicator may be provided in a different location. For example, the or each visual indicator may be provided on the outer surface of the housing 4 in a position which is selectively obscured/exposed by the actuator knob 28. In particular, as shown, the actuator knob 28 comprises a cut away section 29 which provides an ergonomic profile and which exposes a first portion of the housing 4 when in the first configuration and a second portion of the housing 4 when in the second configuration. Accordingly, the first and/or second visual indicators 34a, 34b can be provided on these first and/or section portions.

The housing 4 further comprises a genderless interconnection mechanism. Specifically, the housing 4 comprises a cantilevered beam 36 having an opening 38 (female element) and a complementary retaining hook (i.e., a barb) 40 (male element). The cantilevered beam 36 and retaining hook 40 are provided at diametrically opposed sides of the housing 4.

The actuation knob 28 also comprises a genderless interconnection mechanism. Specifically, the actuation knob 28 comprises a protrusion 44 and a complementary opening 46 which are located side-by-side on its axial end surface 42.

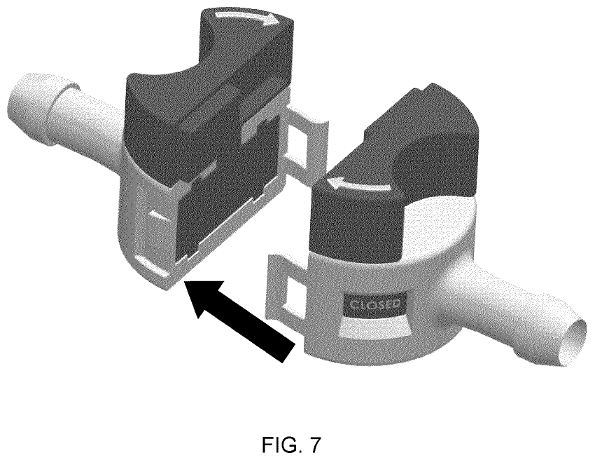

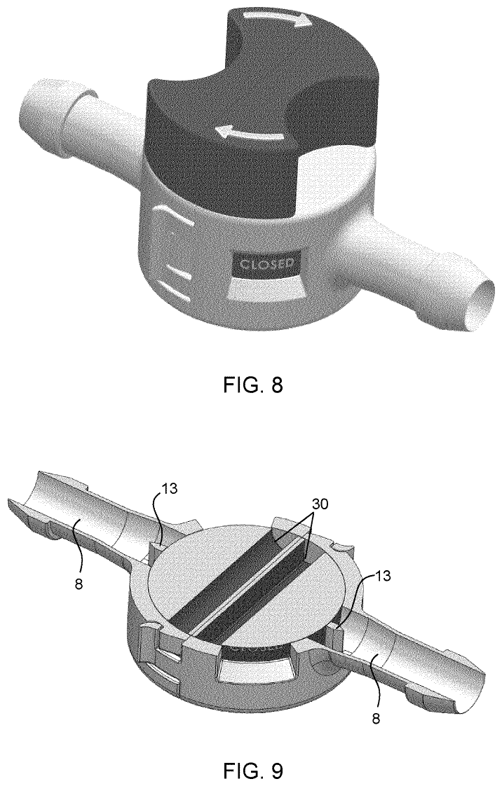

FIGS. 7 and 8 show an assembly comprising a pair of aseptic connectors 2 as described above. The connectors 2 are identical but are referred to as first and second connectors for clarity below, where necessary.

As shown, the first and second connectors are interconnected using the genderless interconnection mechanism provided on the housing 4. Specifically, the hook 40 of the first connector is received in the opening 38 of the second connector and the hook 40 of the second connector is received in the opening 38 of the first connector to form a snap fit connection with the hooks 40 preventing withdrawal. This connects and seals the housings 4 of the connectors 2 to one another.

In a similar manner, the actuator knobs 28 of the first and second connectors 2 are interconnected by virtue of the genderless interconnection mechanism. Specifically, the protrusion 44 of the actuator knob 28 of the first connector is received in the complementary opening 46 of the actuator knob 28 of the second connector and the protrusion 44 of the actuator knob 28 of the second connector is received in the complementary opening 46 of the actuator knob 28 of the first connector. Accordingly, the actuator knobs 28 and thus the rotatable members 14 are constrained to rotate together as one. In other examples, the axial end surfaces 42 may be planar and simply abut against one another such that rotation of one of the actuator knobs 28 causes rotation of the other actuation knob 28.

The connectors 2 are assembled with the rotatable members 14 in the first, closed configuration. As shown in FIG. 9, in this configuration, each passageway 30 is perpendicular to the fluid passageways 8 and thus the ends of the passageway 30 are sealed by the circumferential surface 12 of the housing 4. Similarly, the circumferential surface 16 of the rotatable member 14 seals the opening in the circumferential surface 12. Accordingly, while the connectors 2 are mechanically connected, they remain fluidically disconnected with no fluid flow being possible between their respective fluid passageways 8.

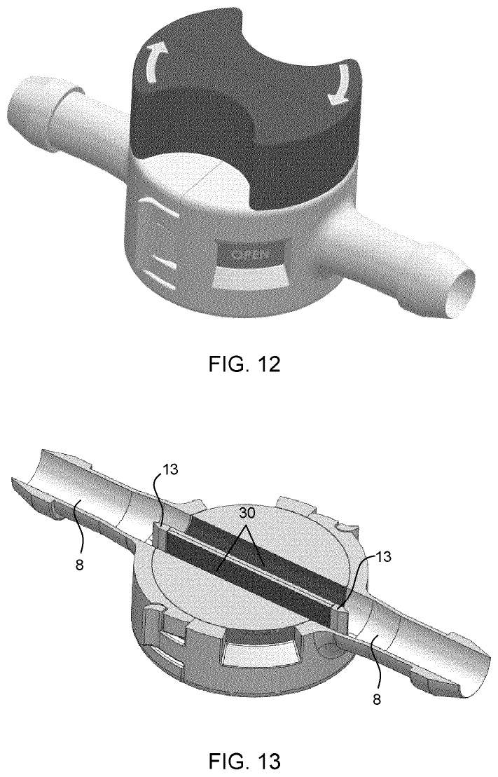

In order to connect their fluid passageways 8, the actuator knobs 28 are grasped and rotated until the ends of the passageways 30 are brought into alignment with the openings of fluid passageways 8 in the circumferential surface 12 of the housings 4. This second, open configuration is shown in FIGS. 12 and 13. A continuous fluid pathway is therefore formed through the first and second connectors 2 such that fluid can pass across the fluid passageways of the connectors 2 via the passageways 30 in the rotatable members 14.

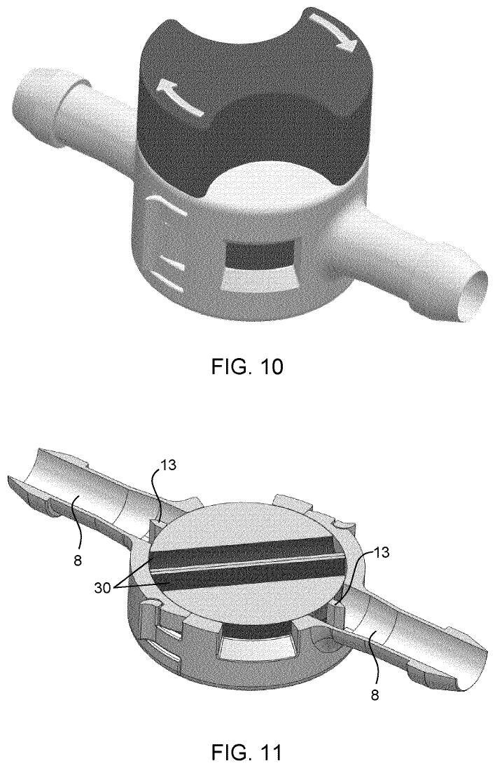

It is worth noting that, during the transition from the first, closed configuration to the second, open configuration, the ends of each passageway 30 remain sealed by the circumferential surface 12 of the housing 4, as shown in FIGS. 10 and 11.

As shown in FIG. 13, in the second, open position, the axial end surfaces 19 of the rotatable members 14 are engaged with one another and are not exposed to the fluid passing through the connectors 2. Accordingly, any contaminants which may have come into contact with the axial end surfaces 19 when they were exposed in the first, open position cannot be introduced into the fluid flow. Further, the portions of the circumferential surface 16 which are adjacent to the axial end surface 19 and thus may be more likely to be exposed to contamination are aligned with the dividing bar 13. Consequently, they are also not exposed to the fluid passing through the connectors 2. The sterility is therefore maintained.

The dividing bar 13 on an inlet side divides the flow of incoming fluid between the passageways 30 and the dividing bar 13 on an outlet side recombines the flow of outgoing fluid after exiting the passageways 30. The dividing bars 13 have a streamlined shape in order to minimise turbulence as the fluid flow is divided and recombined. Specifically, the dividing bars 13 have a width at an outer end which corresponds to the width of the portions of the circumferential surface 16 which are adjacent to the axial end surface 19. The dividing bars 13 taper to a leading edge at an inner end.

The housing 4 and rotatable member 14 may have cooperating catch elements which retain the rotatable member 14 in the closed and/or open configurations and which may be released by user action. For example, the catch elements may retain the rotatable member 14 until a predetermined force is applied to the actuator knob 28 or may require a button or other element to be depressed or otherwise activated to release the rotatable member 14.

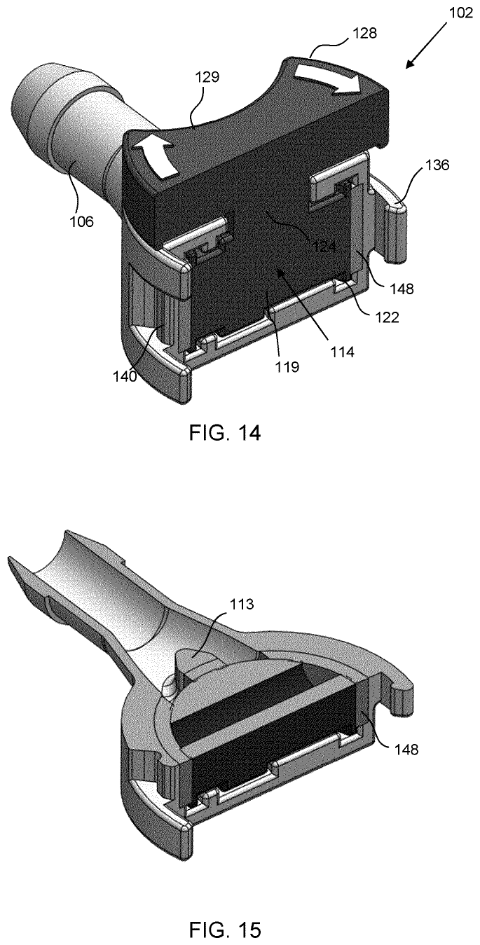

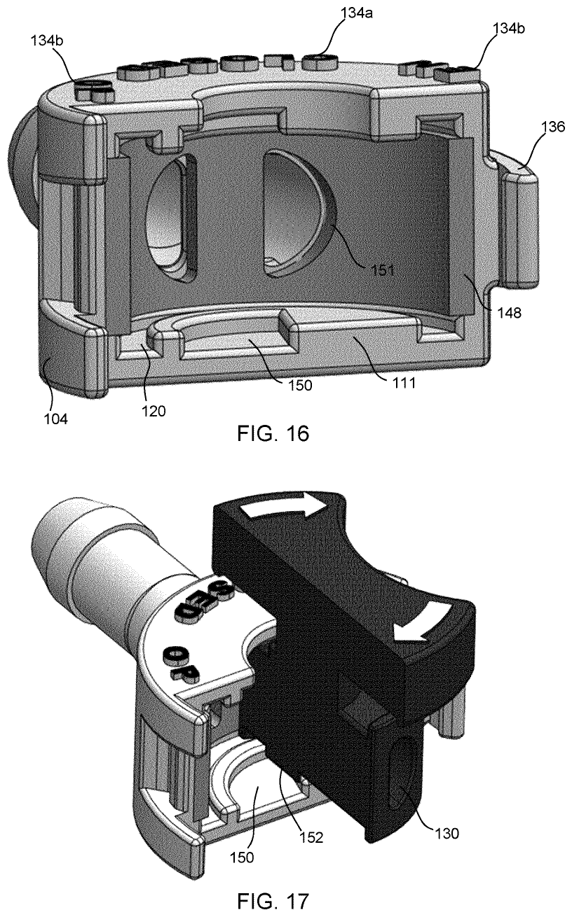

FIGS. 14 to 17 show an aseptic connector 102 according to another embodiment of the disclosure. In this example, the aseptic connector 102 comprises a flexible liner seal 148 which sits within the inner cavity 110 of the housing 104 against the circumferential surface 112, as best shown in FIG. 16. The liner seal 148 comprises a pair of openings 151 which align with the passageway 130 on either side of the dividing bar 113. The liner seal 148 seals against the rotatable member 114 as it rotates relative to the housing 104. The liner seal 148 is affixed to the housing 104 to prevent rotation of the liner seal 148.

As also shown in FIG. 16, the housing 104 of this example comprises a guide slot 150 formed in one or both of the radial end surfaces 111. The guide slot 150 has the form of a quarter circle and receives a tab 152 formed on the rotatable member 114. The tab 152 moves along the guide slot 150 from one end to the other as the rotatable member 114 is rotated by 90 degrees from the closed configuration to the open configuration. In the open configuration, the tab 152 abuts against the end of the guide slot 150 in order to prevent any further rotation of the rotatable member 114.

As shown in FIGS. 16 and 17, in this example, the visual indicators 134a, 134b are formed on the upper surface of the housing 104. The visual indicators 134a, 134b are positioned so as to be selectively obscured/exposed by the actuator knob 128 via the cut away section 129. In particular, the first visual indicator 134a is positioned such that it is visible when the rotatable member 114 is in the first, closed position and the second visual indicator 134b is positioned such that it is visible when the rotatable member 114 is in the second, open position. As shown, the first visual indicator 134a is the word “CLOSED”. The second visual indicator 134b forms the word “OPEN” when the two aseptic connectors 102 are joined together. Specifically, each connector 102 comprises the letters “OP” and the letters “EN” at diametrically opposed positions. When the connectors 102 are joined together, the letters form two instances of the word “OPEN” at diametrically opposed positions.

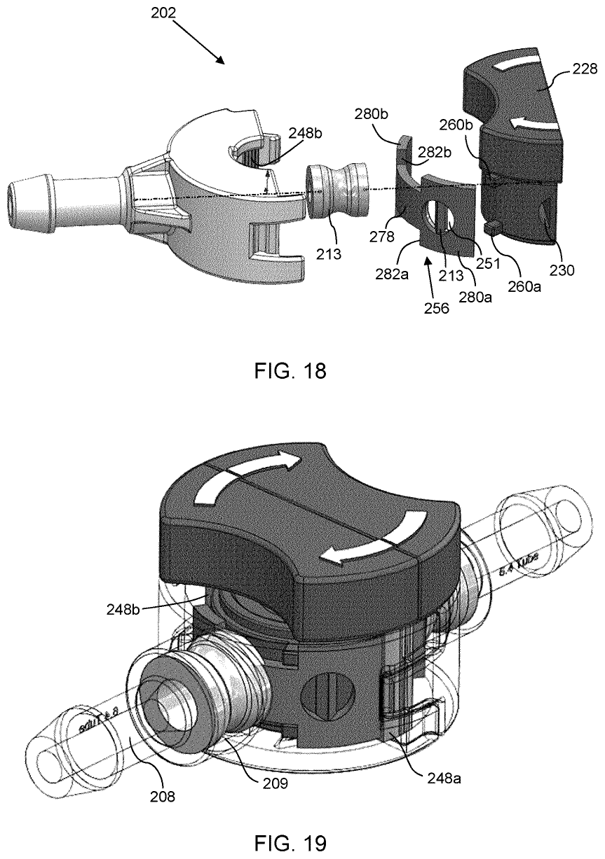

FIGS. 18 to 21 show an aseptic connector 202 according to another embodiment of the disclosure. In this example, the fluid passageway 208 of the aseptic connector 202 does not have a dividing bar. The fluid passageway comprises a counterbore section 209 where it opens into the circumferential surface 212. The counterbore section 209 receives a seal 213. The seal 213 has an hourglass-like profile which comprises a section that tapers (i.e., reduces in diameter) from either end towards the middle in an axial direction. This structure allows the seal 213 to be easily compressed in an axial direction and to return quickly to its relaxed state. The counterbore section 209 has a depth which is less than the length of the seal 213 when in the relaxed state such that the seal 213 partially projects from the counterbore section 209 and is compressed back into the counterbore section 209 by the rotatable members 214.

The seal 213 has a maximum outer diameter which corresponds to an inner diameter of the counterbore section 209 and an inner diameter which corresponds to the inner diameter of the fluid passageway 208. Accordingly, the seal 213 forms a continuation of the fluid passageway 208 with no significant deviation in the diameter.

A pair of liner seals 248a, 248b are affixed to the circumferential surface 212. The liner seals 248a, 248b are provided at either end of the circumferential surface 212 and seal against the circumferential surface 216 of the rotatable member 214. As shown, the liner seals 248a, 248b may be labyrinth seals comprising a plurality of fins which extend in an axial direction.

A sliding seal member 256 is disposed between the circumferential surface 216 of the rotatable member 214 and the circumferential surface 212 of the housing 204. The sliding seal member 256 comprises a central portion 278 and a first and second end portions 280a 280b. The central portion 278 has a reduced width compared to the first and second end portions 280a, 280b such that a pair of first axial abutment shoulders 282a is formed between the central portion 278 and the first end portion 280a and a pair of second axial abutment shoulders 282b is formed between the central portion 278 and the second end portion 280b.

The circumferential surface 216 of the rotatable member 214 comprises a pair of projections 260a, 260b. The central portion 278 of the sliding seal member 256 is received between the pair of projections 260a, 260b with each projection 260a, 260b disposed between an opposing pair of first and second axial abutment shoulders 282a, 282b.

The first end portion 280a comprises a pair of openings 251 which are separate by a dividing bar 213. The openings 251 correspond to the cross-section of the passageways 230.

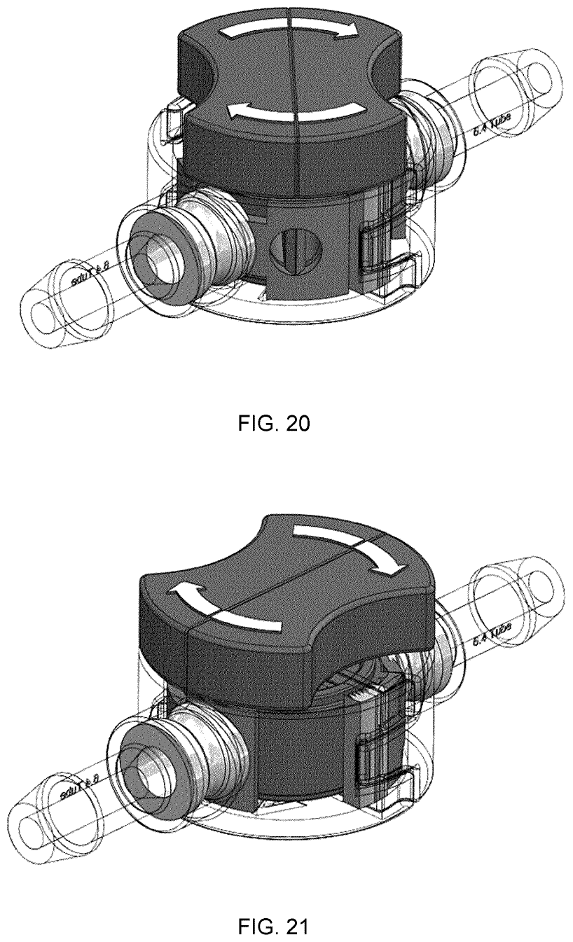

In the closed configuration shown in FIG. 19, the opening at either end of each passageway 230 is covered and sealed by the respective first and second end portions 280a, 280b of the sliding seal member 256. In this position, the opening of the passageway 230 which is covered by the first end portion 280a is offset from the openings 251 formed therein. During the initial rotation of the actuation knob 228, the sliding seal member 256 remains stationary and the projections 260a, 260b translate from a position which is adjacent to the first axial abutment shoulders 282a into engagement with the second axial abutment 282b, as shown in FIG. 20. In this position, adjacent ends of the passageways 230 are aligned with the openings 251 of the sliding seal member 256.

The continued rotation of the actuation knob 228 carries the sliding seal member 256 along the circumferential surface 212 of the housing 204 such that the openings 251 and the ends of the passageways 230 are brought into alignment with the seal 213. This second, open configuration is shown in FIG. 21. A continuous fluid pathway is therefore formed through the first and second connectors 202 such that fluid can pass across the fluid passageways of the connectors 202 via the passageways 230 in the rotatable members 214.

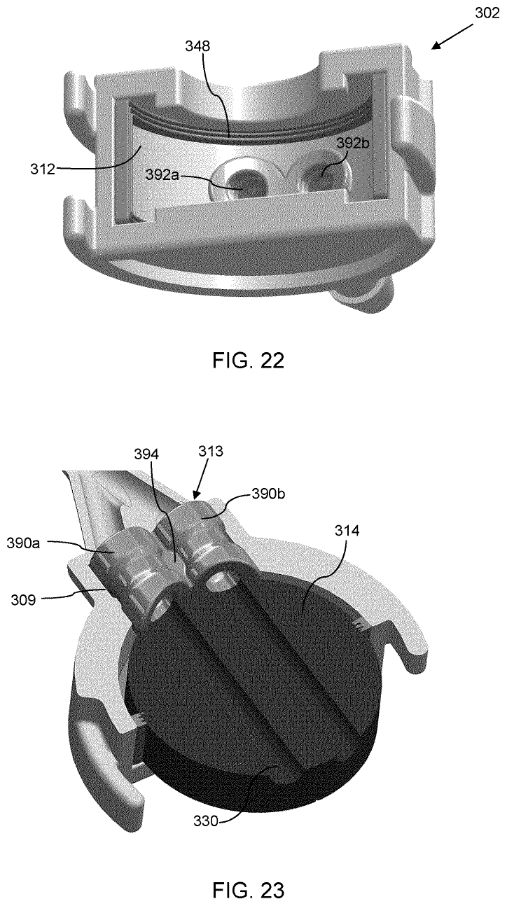

FIGS. 22 and 23 show an aseptic connector 302 according to another embodiment of the disclosure. In this example, a continuous liner seal 348 is provided around the periphery of the circumferential surface 312 of the housing 304. Specifically, the liner seal 348 may be rectangular and affixed to the circumferential surface 312 such that it conforms to the curvature of the circumferential surface 312. The liner seal 348 therefore has first and second axial portions and first and second circumferential portions which form a continuous border around the circumferential surface. The liner seal 348 may be a labyrinth seal which comprises a plurality of fins. The fins may extend in a circumferential direction over the circumferential portions and in an axial direction over the axial portions. The liner seal 348 therefore defines a sterile zone which prevents any contaminants from entering the vicinity of the fluid path.

The aseptic connector 302 also comprises a seal 313 which is received in a complementary bore 309 formed in the housing 304. The seal 313 comprises first and second portions 390a, 390b which have first and second throughbores 392a, 392b.

In this example, each passageway 330 has a circular cross-section, rather than a semi-circular cross-section as shown in the previous embodiments. Each throughbore 392a, 392b therefore has a complementary circular cross-section.

Each of the first and second portions 390a, 390b has an hourglass-like profile which comprises a section that tapers (i.e., reduces in diameter) from either end towards the middle in an axial direction. This structure allows the seal 313 to be easily compressed in an axial direction and to return quickly to its relaxed state. The bore 309 has a depth which is less than the length of the seal 313 when in the relaxed state such that the seal 313 partially projects from the bore 309 and compressed back into the bore 309 by the rotatable members 314. The first and second portions 390a, 390b are joined (i.e., integrally formed) to one another by a web 394 or the like.

The connectors have been described as having a semi-cylindrical cavity and rotatable member which, when connected to an opposing connector, form a combined cylindrical cavity and rotatable member. The combined cylindrical cavity and rotatable member have circular symmetry around an axis of revolution which is aligned with a rotational axis of the rotatable member. It will be appreciated that the combined cavity and rotatable member need not be cylindrical and could be formed by another solid of revolution which has circular symmetry. For example, the combined cavity and rotatable member could be spherical, conical, biconical, etc.

It will be appreciated that in other examples, the connector may have alternative fitting instead of the barb fitting described above. For example, the connector may be provided with a flange fitting.

In other examples, the connector may be provided with a protective cover which seals over the axial end surface of the rotatable member prior to use.

It will be appreciated that the two passageways which form the fluid pathway through the connectors present a larger surface area than a circular bore of the same effective diameter. Action may be taken to avoid any significant increase in frictional losses associated with this. For example, the effective diameter of the fluid pathway formed by the passageways may be increased through the connectors.

To avoid unnecessary duplication of effort and repetition of text in the specification, certain features are described in relation to only one or several aspects or embodiments of the disclosure. However, it is to be understood that, where it is technically possible, features described in relation to any aspect or embodiment of the disclosure may also be used with any other aspect or embodiment of the disclosure.

The disclosure is not limited to the embodiments described herein, and may be modified or adapted without departing from the scope of the present disclosure.

1. An aseptic connector for connection to an identical opposing aseptic connector, the aseptic connector comprising:
a housing having an inner surface which defines a cavity, the housing having a fluid passageway which passes through the inner surface and forms an opening into the cavity;
a rotatable member rotatably mounted within the cavity of the housing and having a fluid passageway extending therethrough;
wherein the rotatable member has a first, closed configuration in which the fluid passageway of the rotatable member is sealed by the inner surface of the housing and a second, open configuration in which the fluid passageway of the rotatable member is aligned with the fluid passageway of the housing and extends to the fluid passageway of the housing of the opposing aseptic connector so as to form a continuous fluid pathway across the aseptic connectors.

2. The aseptic connector as set timed set forth in claim 1, wherein one of the rotatable member and the housing comprises a groove and the other of the rotatable member and the housing comprises a rib which engages with the groove to rotatably mount the rotatable member within the housing.

3. The aseptic connector as set forth in claim 2, wherein the groove and/or rib is semi-circular.

4. The aseptic connector as set forth in claim 1, wherein the rotatable member comprises an actuator portion which extends externally to the housing so as to allow the rotatable member to be rotated between the first and second configurations.

5. The aseptic connector as set forth with in claim 4, wherein the actuator portion comprises a male element and a female element which respectively engage with the female element and the male element of the actuator portion of the opposing aseptic connector.

6. The aseptic connector as set forth in claim 1, wherein the housing of the aseptic connector comprises a male element and a female element which respectively engage with the female element and the male element of the housing of the opposing aseptic connector.

7. The aseptic connector as set forth in claim 6, wherein the male and female elements of the housing form snap fit connections with the female and male elements of the housing of the opposing aseptic connector.

8. The aseptic connector as set forth in claim 1, further comprising a visual indicator which is visible only when the rotatable member is in the first configuration and/or a visual indicator which is visible only when the rotatable member is in the second configuration.

9. The aseptic connector as set forth in claim 8, wherein the or each visual indicator is provided on the rotatable member and the housing comprises a window through which the or each visual indicator can be seen.

10. The aseptic connector as set forth in claim 1, wherein a dividing bar is provided across the opening of the fluid passageway; wherein the dividing bar seals against adjacent portions of the rotatable members of the aseptic connector and the opposing aseptic connector.

11. The aseptic connector as set forth in claim 10, wherein the dividing bar comprises a leading edge which is configured to divide an incoming fluid flow between the fluid passageways of the aseptic connector and the opposing aseptic connector.

12. The aseptic connector as set forth in claim 1, wherein the rotatable member comprises an external surface which faces outwardly when in the first configuration; wherein the external surface abuts and seals against the external surface of the rotatable member of the opposing aseptic connector such that the external surfaces do not form part of the fluid pathway.

13. The aseptic connector as set forth in claim 1, wherein the inner surface of the housing and the rotatable member of the aseptic connector respectively combine with the inner surface of the housing and the rotatable member of the opposing aseptic connector to form a surface of revolution having circular symmetry.

14. The aseptic connector as set forth in claim 1, further comprising a seal member disposed between the housing and the rotatable member; wherein the seal member has an opening which connects the fluid passageway of the housing with the fluid passageway of the rotatable member.

15. The aseptic connector as claimed set forth in claim 14, wherein the seal member is slidable between a first position in which the opening is offset from the fluid passageway of the housing and a second position in which the opening is aligned with the fluid passageway of the housing; wherein the seal member is slidable between the first and second positions when the rotatable member transitions between the first, closed configuration and the second, open configuration.

16. The aseptic connector as set forth in claim 15, wherein in the first, closed configuration the fluid passageway of the rotatable member is sealed by the slidable seal member and wherein the rotatable member rotates relative to the slidable seal member during an initial rotation towards the second, open configuration in order to bring the fluid passageway of the rotatable member into alignment with the opening of the seal member.

17. The aseptic connector as set forth in claim 1, further comprising a liner seal which is disposed between the housing and the rotatable member.

18. The aseptic connector as set forth in claim 17, wherein the liner seal extends around a perimeter of the inner surface of the housing.

19. The aseptic connector as set forth in claim 1, further comprising a seal which is located in a bore formed in the housing and which has a throughbore that forms part of the fluid passageway.

20. The aseptic connector as forth in claim 19, wherein the seal comprises first and second portions which each have a throughbore for connecting to the fluid passageway of the rotatable member of the aseptic connector and the rotatable member of the opposing aseptic connector.

21. The aseptic connector asset forth in claim 20, wherein the first and second portions are integrally formed.

22. This aseptic connector as set forth in claim 21, wherein the first and second portions are joined by a web.

23. The aseptic connector as set forth in claim 20, wherein the first and second portions have hourglass-like profiles.

24. The aseptic connector assembly comprising a pair of aseptic connectors as claimed in claim 1.
